(19) United States
(12) Patent Application Publication
(10) Pub. No.: US 2021/0269885 A1
(43) Pub. Date: Sep. 2, 2021

(54) MULTI-GENE BIOMARKER FOR EARLY DIAGNOSIS OF CANCER

(71) Applicant: BIOINFRA LIFE SCIENCE INC., Seoul (KR)

(72) Inventors: Chul-Woo Kim, Seoul (KR); Ji-Young Jang, Seoul (KR); Yu Jin Park, Seoul (KR)

(21) Appl. No.: 17/260,637

(22) PCT Filed: Jul. 3, 2019
(86) PCT No.: PCT/KR2019/008114

§ 371 Date: Jan. 15, 2021

(30) Foreign Application Priority Data
Jul. 19, 2018 (KR) 10-2018-0084334

(51) Int. Cl. C12Q 1/6886

(52) U.S. Cl. CPC C12Q 1/6886; C12Q 2600/158

(57) ABSTRACT

The present invention relates to a method for early diagnosis of cancer using mRNAs in exosomes, and to a method for early diagnosis of cancer by measuring the expression levels of caveolin-1, adenine nucleotide translocase 2, transforming growth factor-beta 1, and aminoacyl-tRNA synthetase-interacting multifunctional protein-1, and the like.

### TECHNICAL FIELD

The present invention relates to a multi-gene biomarker for early diagnosis of cancer, and more particularly, to a multi-gene biomarker for non-invasive early diagnosis of cancer using an exosome.

### BACKGROUND ART

Globally, the number of cancer patients is increasing year by year, and the number of Korean cancer patients was 100,000 in 2002. Among cancers, breast cancer, stomach cancer, colon cancer, ovarian cancer, liver cancer, prostate cancer, pancreatic cancer, and lung cancer have the highest incidence rates worldwide. The therapeutic efficiency for these cancers varies according to the early diagnosis thereof. Since the early diagnosis of cancer can effectively reduce cancer mortality more than any other therapeutic method, the early diagnosis of cancer is very highly significant in reducing medical costs not only at an individual level but also at a national level.

However, most methods of diagnosing these types of cancer are invasive methods such as biopsy, and thus these methods are very painful and not easily accessible because of side effects of infection and the need for a recovery period after hospitalization and examination, so that examinees generally do not take a test until symptoms appear. Therefore, it is difficult to use these methods for early diagnosis of cancer. Accordingly, in recent years, to diagnose cancer through a non-invasive method, methods of analyzing proteins or DNA from blood, urine, or stool are actively developing. In particular, RNA can be amplified with a small amount, and thus has an advantage of significantly improving diagnostic accuracy. However, since RNA is easily denatured by a large amount of ribonuclease (RNase) present in blood, a diagnostic method using RNA has a disadvantage of decreasing analysis accuracy. In addition, because diagnostic biomarkers are different for each cancer, a large amount of sample and expensive test cost are generated during the test (Korean Patent Application No. 10-2011-0080014).

Therefore, the inventors had attempted to study a method of increasing diagnostic accuracy and reliability by isolation and use of messenger RNA (mRNA) from exosomes in blood that can be protected from RNase, and early diagnosing cancer easily by selecting a minimum amount of multi-gene biomarker that can early diagnose various types of cancer, and thus the present invention was completed.

### DISCLOSURE

### Technical Problem

To solve the above-described problems of the conventional art, the present invention was directed to provide a method of early diagnosing various types of cancer by measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1) using mRNAs in exosomes.

However, technical problems to be solved in the present invention are not limited to the above-described problems, and other problems which are not described herein will be fully understood by those of ordinary skill in the art from the following descriptions.

### Technical Solution

The present invention provides a method of providing information for early diagnosis of cancer, which includes (a) extracting mRNA from an exosome isolated from a biological sample; (b) measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1) using the extracted mRNA as a template; and (c) classifying a case as cancer when all of the mRNA levels of the four genes increase compared with a normal person.

In addition, the present invention provides a composition for early diagnosis of cancer using an exosome, which includes one or more agents for measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1).

In addition, the present invention provides a kit for early diagnosis of cancer, which includes one or more agents for measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1).

In one embodiment of the present invention, the biological sample is preferably obtained by a non-invasive method from blood, urine, or stool, and more preferably, blood. Any sample containing an exosome may be used without limitation. In addition, there is no limitation as long as an exosome is isolated from a sample that can be obtained by a non-invasive method from, for example, blood, urine, or stool.

In another embodiment of the present invention, the cancer may be breast cancer, stomach cancer, colon cancer, ovarian cancer, liver cancer, prostate cancer, pancreatic cancer or lung cancer, or any type of cancer that can be detected with increased expression levels of caveolin-1, adenine nucleotide translocase 2 (ANT2), transforming growth factor-ß1 (TGF-ß1), and aminoacyl-tRNA synthetase-interacting multifunctional proteins-1 (AIMP-1) of the present invention without limitation.

In still another embodiment of the present invention, the one or more agents for measuring the mRNA levels may be a primer or probe that can amplify the mRNAs, or one or more agents for measuring amounts of mRNAs of caveolin-1, adenine nucleotide translocase 2 (ANT2), transforming growth factor-ß1 (TGF-ß1), and aminoacyl-tRNA synthetase-interacting multifunctional proteins-1 (AIMP-1) without limitation.

In yet another embodiment of the present invention, the composition or kit may further include an agent for measuring mRNA level(s) of 18S rRNA and/or B-actin, and an agent that can be additionally included as long as it is a housekeeping mRNA that can be used as an internal control without limitation. In addition to this, it may further include an agent for measuring mRNA for a gene known to be able to diagnose cancer early. In addition, it may further include an agent for lysing an exosome, an agent for isolating mRNA, etc. to isolate mRNA in an exosome.

### Advantageous Effects

Since a biomarker group consisting of caveolin-1, adenine nucleotide translocase 2 (ANT2), transforming growth factor-ß1 (TGF-ß1), and aminoacyl-tRNA synthetase-interacting multifunctional proteins-1 (AIMP-1) according to the present invention is an easily accessible, non-invasive diagnostic method using mRNA in an exosome, and thus can early diagnose various types of cancer with high accuracy, the early diagnosis rate of cancer can be considerably increased, and thereby, it is expected to considerably increase cancer therapeutic efficiency. In addition, it is expected to be able to increase therapeutic efficiency against recurrence by easily monitoring recurrence after cancer treatment.

### DESCRIPTION OF DRAWINGS

FIGS. 1A and 1B show the results of selecting a multi-gene biomarker for early diagnosis of cancer using qRT-PCR according to one exemplary embodiment of the present invention.

FIGS. 2A and 2B show the results of selecting a multi-gene biomarker for early diagnosis of cancer by revalidation of the multi-gene biomarker for early diagnosis of cancer selected using qRT-PCR according to one exemplary embodiment of the present invention.

### MODES OF THE INVENTION

The most effective method for reducing cancer mortality is expected to substantially reduce cancer recurrence or mortality by diagnosing cancer early.

In the present invention, a variety of cancers may be diagnosed early with high accuracy by a non-invasive method by measuring an mRNA level of a multi-gene biomarker consisting of caveolin-1, adenine nucleotide translocase 2 (ANT2), transforming growth factor-ß1 (TGF-ß1), and aminoacyl-tRNA synthetase-interacting multifunctional proteins-1 (AIMP-1) using mRNA in an exosome, and therefore, the biomarker can be expected to be effectively used in early diagnosis of cancer.

The term “exosome” used herein broadly includes small-sized vesicles having a lipid bilayer, which are released from cells, and exosomes are known to be released from various cells and have a diameter of approximately 30 to 200 nm. Such an exosome includes various types of proteins, DNA, mRNA, and miRNA derived from a cell, and it is preferable that the exosome of the present invention is naturally or artificially secreted, or prepared.

The term “biological sample” used herein refers to a sample obtained non-invasively, and may include all samples containing an exosome, preferably, blood, plasma, serum, bone marrow, tissue, cell, saliva, sputum, hair, urine or stool, and more preferably, blood, urine or stool. Any sample that can measure an mRNA level of a multi-gene biomarker consisting of caveolin-1, adenine nucleotide translocase 2 (ANT2), transforming growth factor-ß1 (TGF-ß1), and aminoacyl-tRNA synthetase-interacting multifunctional proteins-1 (AIMP-1) is used without limitation.

The “method for providing information” used herein is a method of providing information on early diagnosis of cancer, and also refers to a method of acquiring information on the possibility of cancer occurrence when mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1) increase using mRNA in an exosome.

The “method of measuring mRNA levels” used herein is a process of confirming the presence of mRNAs of caveolin-1, adenine nucleotide translocase 2 (ANT2), transforming growth factor-ß1 (TGF-ß1), and aminoacyl-tRNA synthetase-interacting multifunctional proteins-1 (AIMP-1) genes from an exosome and mRNA amounts thereof to diagnose cancer, by measuring the amounts of mRNAs included in an exosome. As the method of analyzing the mRNA levels, RT-PCR, competitive RT-PCR, real-time RT-PCR, RNase protection assay (RPA), northern blotting, a DNA microarray chip, and Target-Specific PCR sequence detection with MagPlex®-TAG™ Microsphere using Luminex may be used, but the present invention is not limited thereto.

The “kit” used herein refers to a tool for examination which can predict the occurrence of disease by measuring mRNA amounts of caveolin-1, adenine nucleotide translocase 2 (ANT2), transforming growth factor-ß1 (TGF-ß1), and aminoacyl-tRNA synthetase-interacting multifunctional proteins-1 (AIMP-1) genes in an exosome. Any tool that can measure the mRNA amounts of the genes from a biological sample isolated from a patient is used without limitation. The kit preferably includes a probe or primer set having a complementary sequence with respect to the mRNA, and the “probe” or “primer” used herein refers to an oligonucleotide having a sequence complementary to the mRNA, and any agent for measuring the mRNA amounts of the genes is used without limitation.

Hereinafter, to help in understanding the present invention, exemplary examples will be suggested. However, the following examples are merely provided to more easily understand the present invention, and not to limit the present invention.

### EXAMPLES

### Example 1: Preparation of Blood Samples from Normal Persons and Cancer Patients

To select a multi-gene biomarker for early diagnosis of cancer, blood samples from normal patients, and cancer patients were prepared. The samples from cancer patients were blood samples from patients diagnosed with cancer in a university hospital, and the samples from normal persons were blood samples from persons within a normal range as a result of examination using a biomarker for early diagnosis of cancer. Each sample was stored in a refrigerator at −80□, and serum collected in a vacutainer blood collection tube such as a serum separator tube (SST) and a plasma collected in ethylenediaminetetraacetic acid (EDTA) vacutainer blood collection tube was used. To extract RNA in an exosome contained in each sample, a pooling sample was prepared as shown in Table 1 below. The prepared pooling sample was dispensed into fresh 200-μl tubes, and stored in a refrigerator at −80□, and each sample was subjected to a single process of freezing and thawing.

TABLE 1

| | | |
|---|---|---|
| Serum of normal | Male | 200 μl each of five samples were pooled to prepare a 1 ml sample |
| person | Female | 200 μl each of five samples were pooled to prepare a 1 ml sample |
| Plasma of normal | Male | 200 μl each of five samples were pooled to prepare a 1 ml sample |
| person | Female | 200 μl each of five samples were pooled to prepare a 1 ml sample |
| Serum of breast | Stage 1 & 2 | 200 μl each of five samples were pooled to prepare a 1 ml sample |
| cancer | Stage 3 & 4 | 200 μl each of five samples were pooled to prepare a 1 ml sample |
| Serum of stomach | Stage 1 & 2 | 200 μl each of five samples were pooled to prepare a 1 ml sample |
| cancer | Stage 3 & 4 | 200 μl each of five samples were pooled to prepare a 1 ml sample |
| Serum of colon | Stage 1 & 2 | 200 μl each of five samples were pooled to prepare a 1 ml sample |
| cancer | Stage 3 & 4 | 200 μl each of five samples were pooled to prepare a 1 ml sample |
| Serum of ovarian | Stage 1 & 2 | 200 μl each of five samples were pooled to prepare a 1 ml sample |
| cancer | Stage 3 & 4 | 200 μl each of five samples were pooled to prepare a 1 ml sample |
| Serum of liver | Stage 1 & 2 | 200 μl each of Five samples were pooled to prepare a 1 ml sample |
| cancer | stage 3 & 4 | 200 μl each of five samples were pooled to prepare a 1 ml sample |
| Serum of prostate | stage 1 & 2 | 200 μl each of five samples were pooled to prepare a 1 ml sample |
| cancer | stage 3 & 4 | 200 μl each of five samples were pooled to prepare a 1 ml sample |
| Plasma of pancreatic | stage 1 & 2 | 200 μl each of five samples were pooled to prepare a 1 ml sample |
| cancer | stage 3 & 4 | 200 μl each of five samples were pooled to prepare a 1 ml sample |
| Plasma of lung | stage 1 & 2 | 200 μl each of five samples were pooled to prepare a 1 ml sample |
| cancer | stage 3 & 4 | 200 μl each of five samples were pooled to prepare a 1 ml sample |

### Example 2: Extraction of RNA in Exosome

To extract messenger RNA (mRNA) in an exosome from the pooling sample prepared by the method described in Example 1, Nextractor® NX-48 (Genolution) was used. In further detail, exosomes in plasma or serum were lysed using Nextractor® NX-48, and only RNAs were purely isolated from various proteins, DNAs, and RNAs (mRNA, miRNA, rRNA, etc.) contained in the exosomes. The extracted RNAs were subjected to measurement of absorbance at 260 nm using NanoDrop to quantify RNA concentrations, and RNA purities and contamination degrees were confirmed using a 260/280 ratio and a 260/230 ratio.

### Example 3: Selection of Multi-Gene Biomarker for Early Diagnosis of Cancer

3.1. Primary Selection of Multi-Gene Biomarker for Early Diagnosis of Cancer

To select a multi-gene biomarker for early diagnosis of cancer, 100 ng of RNA extracted by the same method as described in Example 2 was used as a template and subjected to quantitative reverse transcription-polymerase chain reaction (qRT-PCR). The qRT-PCR was conducted using a SensiFAST™ Probe Lo-ROX One-Step kit (Bioline) and a StepOne Plus system (ABI). As candidates for multi-gene biomarkers for early diagnosis of cancer, primarily, 25 candidate genes were selected by examining (a) genes involved in tumor proliferation, (b) genes involved in tumor progression, (c) genes involved in tumor immune responses, and (d) genes involved in cancer stem cells, and subjected to qRT-PCR using cancer cell line-derived exosomes.

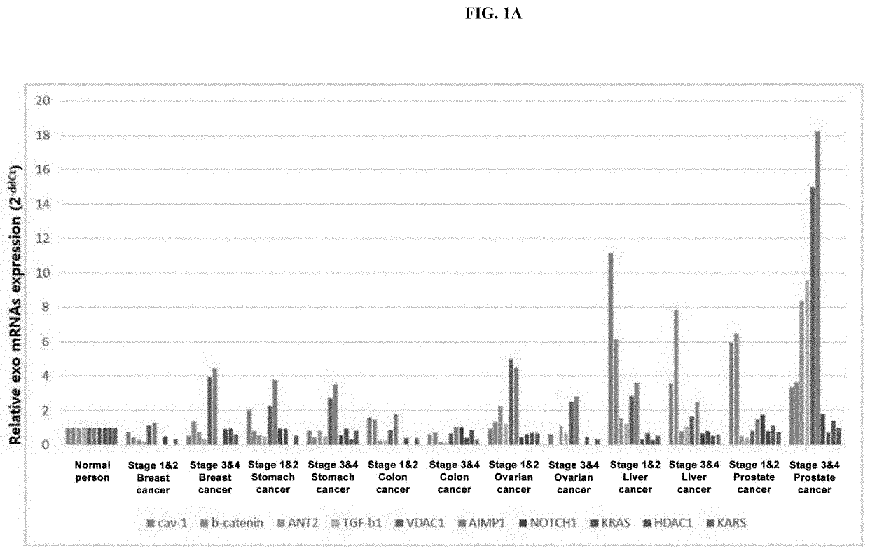

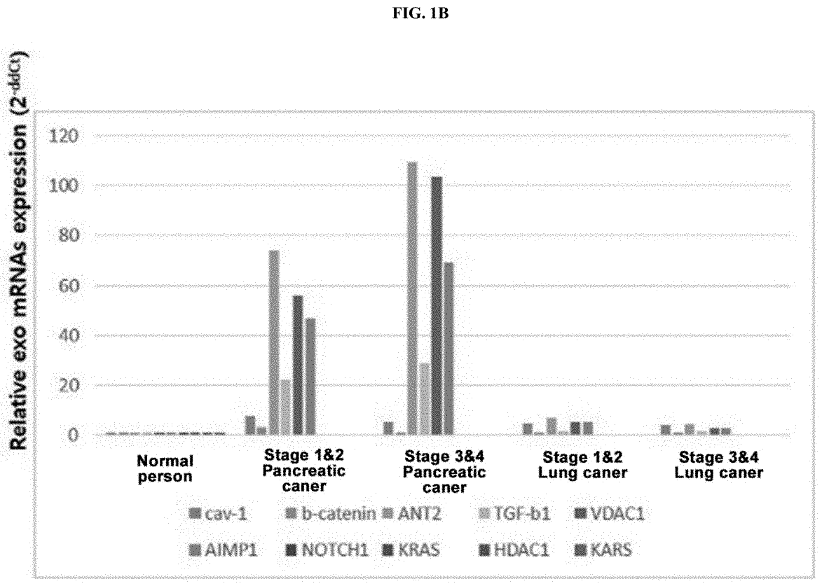

As a result of the qRT-PCR on the cancer cell line-derived exosomes, it was confirmed that amounts of 14 mRNAs of caveolin-1, B-catenin, adenine nucleotide translocase 2 (ANT2), voltage-dependent anion channel 1 (VDAC1), transforming growth factor-ß1 (TGF-ß1), aminoacyl-tRNA synthetase-interacting multifunctional proteins-1 (AIMP-1), NOTCH 1, v-Ki-ras2 Kirsten rat sarcoma viral oncogene homolog (KRAS), histone deacetylase 1 (HDAC1), lysyl-tRNA synthetase (KARS), transforming growth factor-ß3 (TGF-ß3), cancer antigen 125 (CA-125), ecto-NOX disulfide-thiol exchanger 2 (ENOX2) and a carcinoembryonic antigen (CEA) increase in the 25 candidate genes. Targeting the 14 genes, the mRNAs extracted from the cancer patient samples prepared by the same methods as described in Examples 1 and 2 were used as templates, and subjected to qRT-PCR. As an internal control gene for normalization of an RNA concentration, B-actin, and 18S rRNA were used as housekeeping genes. Probe information for each gene is shown in Table 2 below. When the qRT-PCR was performed on 100 ng of RNA, except TGF-ß3, CA125, ENOX2, and CEA genes having a Ct value of 35 or more, the amounts of mRNAs were measured. The result is shown in FIG. 1.

| | |
|---|---|
| Gene name | IDT cat no. (assay name) |

Target gene

| | | |
|---|---|---|
| \textcircled{1} | Caveolin-1 | Hs.PT.56a.40058555.g |
| \textcircled{2} | β-catenin | Hs.PT.58.3699397 |
| \textcircled{3} | ANT2 | Hs.PT.56a.39886014.g |
| \textcircled{4} | VDAC1 | Hs.PT.58.2339327.g |
| \textcircled{5} | TGF-β1 | Hs.PT.58.27299467.g |
| \textcircled{6} | AIMP-1 | Hs.PT.58.26283651.g |
| \textcircled{7} | NOTCH1 | Hs.PT.58.41121293.g |
| \textcircled{8} | KRAS | Hs.PT.58.3054425.g |
| \textcircled{9} | HDAC1 | Hs.PT.58.28348857.g |
| \textcircled{10} | KARS | Hs.PT.58.27546126.g |
| \textcircled{11} | TGF-β3 | Hs.PT.58.27186053.g |
| \textcircled{12} | CA125 | Hs.PT.58.2308101 |
| \textcircled{13} | ENOX2 | Hs.PT.58.145266 |
| \textcircled{14} | CEA | Hs.PT.58.39176897.g |

Internal control gene

| | | |
|---|---|---|
| \textcircled{1} | β-actin | Hs.PT.56a.40703009.g |
| \textcircled{2} | 18S rRNA | Hs.PT.39a.22214856.g |

As shown in FIG. 1, based on the expression level in a normal person, six genes of Caveolin-1, ß-catenin, ANT2, VDAC1, TGF-ß1, and AIMP-1, which are mRNAs significantly increasing two-fold or more, were selected. Among these genes, ANT2 and VDAC1 are known to be involved in tumor proliferation, AIMP-1 is known to be involved in tumor progression, TGF-ß1 is known to be involved in tumor immune responses, and Caveolin-1 and ß-catenin are known to be involved in cancer stem cells.

3.2. Revalidation of Multi-Gene Biomarker for Early Diagnosis of Cancer

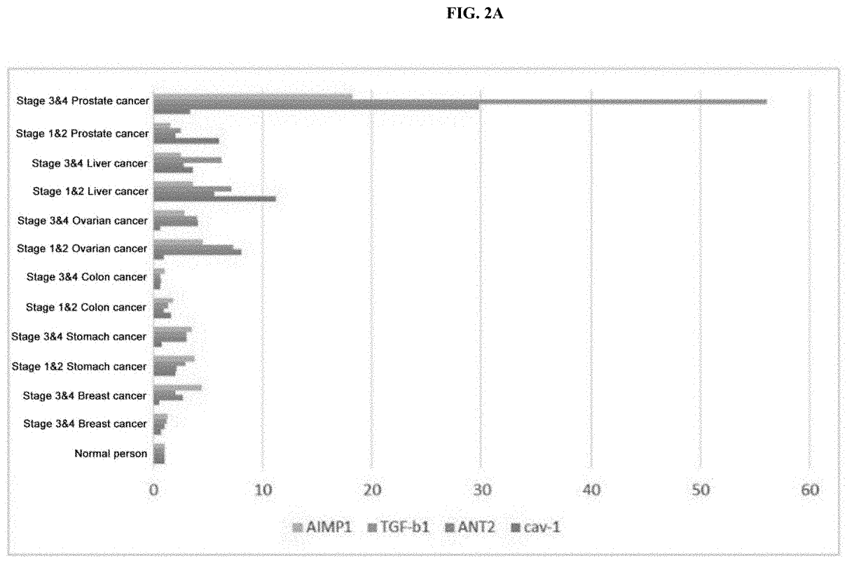

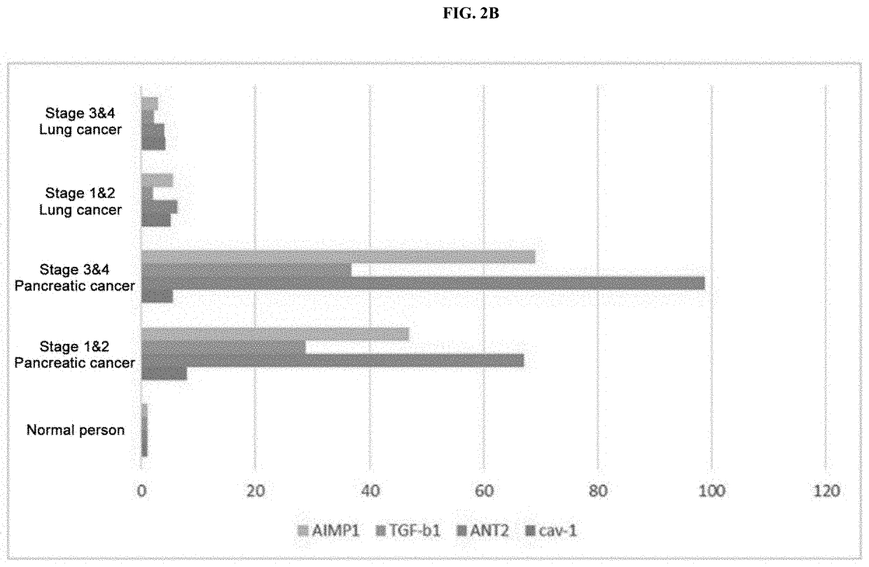

To revalidate whether the six genes selected by the method described in Example 3.1 can be used as multi-gene biomarkers for early diagnosis of a variety of cancers, qRT-PCR was performed on the six genes using mRNA isolated from a cancer patient-derived exosome as a template. The result is shown in FIG. 2.

As shown in FIG. 2, in the case of a multi-gene biomarker used by combining four genes such as AIMP-1, TGF-ß1, ANT2, and Caveolin-1, rather than a multi-gene biomarker prepared by a combination of six genes, it was able to be confirmed that the accuracy of early diagnosis of various types of cancer such as breast cancer, colon cancer, stomach cancer, ovarian cancer, liver cancer, prostate cancer, pancreatic cancer, and lung cancer is improved.

According to the results, among the combinations of various genes known to be related to cancer, it is expected that a combination of AIMP-1, TFG-ß1, ANT2, and Caveolin-1 according to the present invention may not only significantly increase the accuracy of early diagnosis of various types of cancer, but also significantly improve the efficiency of early diagnosis of cancer because there is easy access by a non-invasive diagnosis method using an exosome in blood.

The above-described description of the present invention is merely provided to exemplify the present invention, and it should be understood by those of ordinary skill in the art to which the present invention belongs that the present invention can be implemented in modified forms without departing from the essential features of the present invention. Therefore, the exemplary embodiments described above should be interpreted as illustrative and not limited in any aspect.

### INDUSTRIAL APPLICABILITY

The present invention relates to a method of early diagnosing cancer using mRNA in an exosome, and since this method can be used not only to diagnose various types of cancer early with high accuracy by a non-invasive method using mRNA in an exosome, but also to monitor recurrence after treatment, it is expected that this method may reduce cancer treatment costs and unnecessary testing costs after treatment due to the increase in early cancer diagnosis rate.

1. A method of providing information for early diagnosis of cancer, comprising:
(a) extracting mRNA from an exosome isolated from a biological sample;
(b) measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1) using the extracted mRNA as a template; and
(c) classifying a case as cancer when all of the mRNA levels of the four genes increase compared with a normal person.

2. The method of claim 1, wherein the biological sample is blood, urine, or stool.

3. The method of claim 1, wherein the cancer is any one selected from the group consisting of breast cancer, stomach cancer, colon cancer, ovarian cancer, liver cancer, prostate cancer, pancreatic cancer, and lung cancer.

4. A composition for early diagnosis of cancer using an exosome, comprising:
one or more agents for measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1).

5. The composition of claim 4, wherein the one or more agents for measuring mRNA levels are a primer or probe that can amplify the mRNAs.

6. The composition of claim 4, wherein the exosome is isolated from blood, urine, or stool.

7. The composition of claim 4, wherein the cancer is any one selected from the group consisting of breast cancer, stomach cancer, colon cancer, ovarian cancer, liver cancer, prostate cancer, pancreatic cancer, and lung cancer.

8. The composition of claim 4, wherein the composition further comprises an agent for measuring an mRNA level of 18S rRNA or B-actin.

9. A kit for early diagnosis of cancer using an exosome, comprising:
one or more agents for measuring mRNA levels of caveolin-1, adenine nucleotide translocase 2 (ANT2), transforming growth factor-ß1 (TGF-ß1), and aminoacyl-tRNA synthetase-interacting multifunctional proteins-1 (AIMP-1).

10. The kit of claim 9, wherein the one or more agents for measuring mRNA levels are a primer or probe that can amplify the mRNAs.

11. The kit of claim 9, wherein the exosome is isolated from blood, urine, or stool.

12. The kit of claim 9, wherein the cancer is any one selected from the group consisting of breast cancer, stomach cancer, colon cancer, ovarian cancer, liver cancer, prostate cancer, pancreatic cancer, and lung cancer.

13. The kit of claim 9, wherein the kit further comprises an agent for measuring an mRNA level of 18S rRNA or ß-actin.

14. The kit of claim 9, wherein the kit further comprises an agent for isolating mRNA from the exosome.
